(19) United States
(12) Patent Application Publication
(10) Pub. No.: US 2013/0237983 A1
(43) Pub. Date: Sep. 12, 2013

(54) Patient-Warming Apparatus Including an Electrosurgical Return Electrode

(71) Applicants: Andrew Giles, Libertyville, IL (US); Francis A. Czajka, JR., Libertyville, IL (US); Benjamin Duck, Johnsburg, IL (US)

(72) Inventors: Andrew Giles, Libertyville, IL (US); Francis A. Czajka, JR., Libertyville, IL (US); Benjamin Duck, Johnsburg, IL (US)

(21) Appl. No.: 13/415,426

(22) Filed: Mar. 8, 2012

(51) Int. Cl. A61B 18/14; H05B 3/00

(52) U.S. Cl. 606/41; 29/611

(57) ABSTRACT

A patient-warming apparatus integrally comprises a patient-support substrate, a patient-heating element disposed proximal to the patient-support substrate, and an electrosurgical return electrode that is also disposed proximal to the patient-support substrate. By one approach the electrosurgical return electrode is integral to the patient-heating element. By another approach the electrosurgical return electrode is disposed between the patient-support substrate and the patient-heating element. In all of these cases, if desired, the electrosurgical return electrode comprises a part of a capacitively-coupled electrosurgical return electrode.

### TECHNICAL FIELD

This invention relates generally to patient warming and to electrosurgery.

### BACKGROUND

Providing local warming to a patient during, for example, surgery comprises a known area of endeavor. In addition to passive mechanisms (such as blankets to dispose over a patient) active mechanisms are sometimes used to provide local, controlled heating to portions of a patient's body. Such active mechanisms include, for example, electric heating elements that convert electricity into heat. While sometimes employed to improve the patient's comfort, patient warming can also provide important patient treatment and/or recovery support as well.

Electrosurgery is also known in the art. Electrosurgery comprises the use of radio-frequency energy (often in the range of 200 KHz to 4 MHz) as administered via a hand-held tool to both cut and cauterize a patient's tissue during surgical procedures. Electrosurgery typically relies upon using the patient's own body as a part of the electrical energy circuit.

There are times when it may be desirable to utilize both active patient warming and electrosurgery for a given patient during a given procedure. Unfortunately, present practices in these two regards tend to present obstacles in these regards. In particular, in many cases effective use of one practice can be significantly impaired when also using the other practice.

### BRIEF DESCRIPTION OF THE DRAWINGS

The above needs are at least partially met through provision of a patient-warming apparatus that includes an electrosurgical return electrode as described in the following detailed description, particularly when studied in conjunction with the drawings, wherein:

FIG. 1 comprises a side-elevational schematic view as configured in accordance with various embodiments of the invention;

FIG. 2 comprises a bottom perspective view as configured in accordance with various embodiments of the invention;

FIG. 3 comprises a side-elevational schematic view as configured in accordance with various embodiments of the invention;

FIG. 4 comprises a side-elevational schematic view as configured in accordance with various embodiments of the invention;

FIG. 5 comprises a top plan schematic view as configured in accordance with various embodiments of the invention; and

FIG. 6 comprises a top perspective view as configured in accordance with various embodiments of the invention.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein.

### DETAILED DESCRIPTION

Generally speaking, pursuant to these various embodiments a patient-warming apparatus integrally comprises a patient-support substrate, a patient-heating element disposed proximal to the patient-support substrate, and an electrosurgical return electrode that is also disposed proximal to the patient-support substrate. By one approach the electrosurgical return electrode is integral to the patient-heating element. By another approach the electrosurgical return electrode comprises a physically-discrete component and is disposed, for example, between the patient-support substrate and the patient-heating element. In all of these cases, if desired, the electrosurgical return electrode comprises a part of a capacitively-coupled electrosurgical return electrode.

By one approach the patient-support substrate comprises a non-gel patient-support substrate (comprised, for example, of memory foam).

By one approach the patient-warming apparatus also includes one or more temperature sensors. In this case, if desired, a corresponding patient-warming control circuit can use the temperature sensor(s) to control the patient-heating element to compensate, for example, for heat exuded by the electrosurgical return electrode. This can result in energy savings as well as increased patient comfort and/or safety.

So configured, a medical-services provider need not be forced to choose between actively warming the patient and utilizing electrosurgical techniques when providing services to that patient. Instead, both capabilities are integrally supported in a manner that permits both to function in an appropriate manner. These teachings are readily employed with existing warming and electrosurgical approaches and hence can serve to greatly leverage the continued value and viability of known methodologies in these regards. These teachings are also highly flexible and scalable in practice and will accommodate a wide variety of variations in practice to suit the needs and/or opportunities that tend to characterize a given application setting.

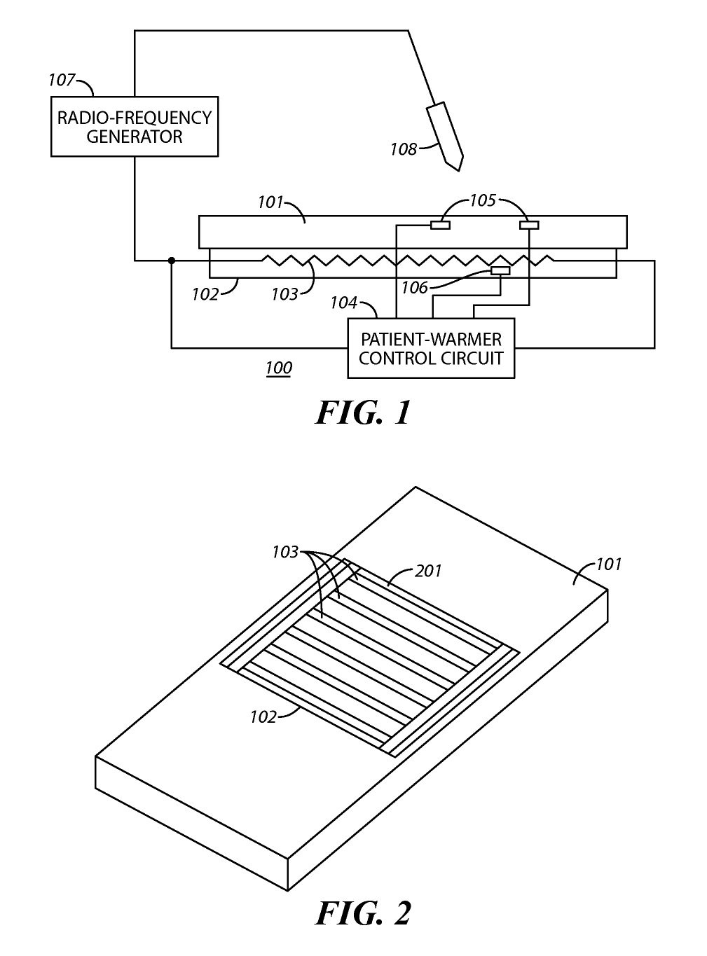

These and other benefits may become clearer upon making a thorough review and study of the following detailed description. Referring now to the drawings, and in particular to FIG. 1, an illustrative patient-warming apparatus 100 that is compatible with many of these teachings will now be presented.

In this illustrative example the patient-warming apparatus 100 includes a patient-support substrate 101. This patient-support substrate 101 can comprise, by one approach, a non-gel material such as but not limited to so-called memory foam. (While gel materials might be suitable in some specific application settings, gel tends to exhibit considerable thermal inertia. This thermal inertia, in turn, can slow the initial delivery of warming heat to a patient and can also make it more difficult to maintain a specific targeted level of warmth over time.)

By one approach this patient-support substrate 101 is rectangular in shape and is of sufficient length and width to support at least the head, torso, and legs of a patient. The thickness of the patient-support substrate 101 can vary as desired. For many application settings the thickness of the patient-support substrate 101 can range from about 1 inch to about 6 inches with other thicknesses being possible.

The patient-warming apparatus 100 also includes a patient-heating element 102. This patient-heating element 102 is disposed proximal to the patient-support substrate 101. In this illustrative example the patient-heating element 102 is directly adjacent to the patient-support substrate 101. Referring momentarily to both FIGS. 1 and 2, this patient-heating element 102 comprises a plurality of electrically-resistive traces 103 (formed, for example, of copper or a copper alloy) that are disposed (for example, via a printing, sputtering, or other deposition process) on a flexible substrate 201 (such as, for example, a polyimide film such as DuPont's Kapton material). The dimensions of these electrically-resistive traces 103 as well as their length and number can vary with the intended application setting as desired. The fabrication of such a patient-heating element 102 is known in the art and therefore further elaboration will not be provided here in these regards.

In the illustrative examples provided in FIGS. 1 and 2, the patient-heating element 102 is disposed on a side of the patient-support substrate 101 that is opposite the side upon which a patient rests (FIG. 2 comprising a bottom perspective view). The size of the patient-heating element 102 relative to the patient-support substrate 101 can vary with the intended application setting. FIG. 1, for example, depicts the patient-heating element 102 as being largely coextensive with the patient-support substrate 101 whereas FIG. 2 depicts the patient-heating element 102 as being only about one third the size of the patient-support substrate 101.

For the sake of simplicity and clarity only a single patient-heating element 102 is shown. If desired, however, a given patient-warming apparatus 100 can comprise a plurality of patient-heating elements. In such a case the various patient-heating elements can all be essentially identical to one another or can, as desired, vary from one another with respect to their relative dimensions and/or shape, composition, and so forth.

With continued reference to FIG. 1, the patient-warming apparatus 100 can further include a patient-warmer control circuit 104. This patient-warmer control circuit 104 operably couples to the patient-heating element 102 and is configured to control the warming behavior of the patient-heating element 102. Such a control circuit 104 can comprise a fixed-purpose hard-wired platform or can comprise a partially or wholly programmable platform. All of these architectural options are well known and understood in the art and require no further description here.

In this illustrative example the patient-warmer control circuit 104 operably couples to a pair of temperature sensors 105 that are disposed within the patient-support substrate 101 and which each serve to sense a local temperature that corresponds to the heat being locally delivered to the patient. The patient-warmer control circuit 104 also operably couples to another temperature sensor 106 disposed as part of the patient-heating element 102 and which serves to sense the temperature thereof.

The patient-warmer control circuit 104 is configured to control the patient-heating element 102 as a function, at least in part, of the information provided by these sensors 105 and 106. This can comprise, for example, bringing the delivery of heat to the patient to a given target temperature and then maintaining that target temperature within some predetermined range (such as one or two degrees Celsius). (The patient-warmer control circuit 104 can also couple to a user interface (not shown) if desired to provide a mechanism by which, for example, an end user can set one or more target temperatures and/or permitted temperature ranges. Such a user interface can also include a display to thereby provide, for example, temperature information to the end user.)

So configured, a patient resting atop the patient-support substrate 101 can be warmed as desired. The illustrated apparatus 100, however, also includes an electrosurgical return electrode that operably couples to a radio-frequency generator 107 that also couples to the surgical tool 108 that serves as the active electrode for the corresponding electrosurgical system. Electrosurgical systems are well known in the art and, as the present teachings are not particularly sensitive to any particular approaches in these regards, further elaboration will not be provided here regarding such systems save to note that this particular electrosurgical system comprises a system that utilizes a capacitively-coupled electrosurgical return electrode.

In the approach illustrated in FIG. 1, the patient-heating element 102 and the electrosurgical return electrode share at least one common electricity-conveying component and are, in fact, one and the same. That is to say, the patient heating element 102 also electrically couples to the radio-frequency generator 107 and serves as one plate of a coupling capacitor where the patient comprises the other plate of the coupling capacitor.

So configured, the patient-warming apparatus 100 can simultaneously warm a patient in a controlled manner while also providing an electrical return path in support of electrosurgery.

Electrosurgery return electrodes typically exude heat during use. By one approach, if desired, the foregoing temperature sensors 105 and/or 106 (or one or more temperature sensors located elsewhere as desired) can be utilized by the patient-warmer control circuit 104 to also control the patient-heating element 102 to compensate, at least in part, for such heat exuded by the electrosurgical return electrode. This can comprise, for example, reducing the heat provided by the patient-heating element 102 to compensate for heat being given off by the electrosurgical return electrode.

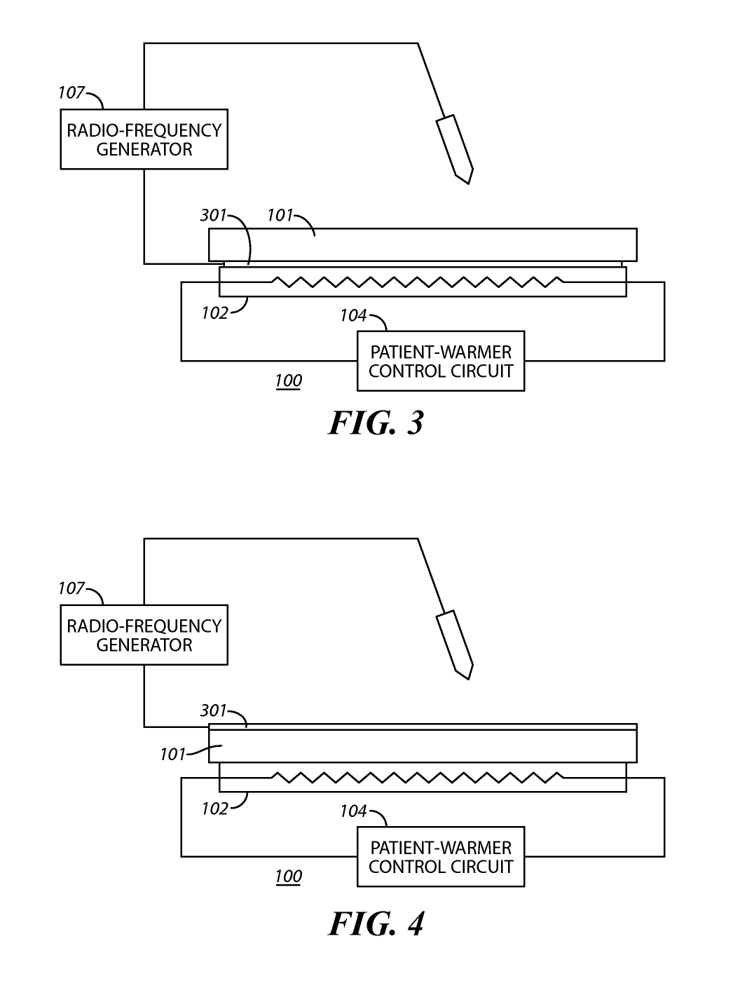

In the example above the patient-heating element 102 also serves as the electrosurgical return electrode. These teachings, however, are very flexible in these regards. In FIG. 3, for example, the patient-heating element 102 and the electrosurgical return electrode 301 are physically-separate discrete components. In this case the electrosurgical return electrode 301 can comprise any of a variety of electrically conductive materials including, for example, a metal foil (such as an aluminum foil) or mesh, an electrically-conductive fabric (such as a medical-grade conductive elastic fabric), a planar conductor disposed on a flexible substrate, a metal plate, and so forth.

In this example the electrosurgical return electrode 301 is disposed between the patient-support substrate 101 and the patient-heating element 102. In this illustrative example these components are disposed directly adjacent one another. By one approach, the electrosurgical return electrode 301 is substantially coextensive with the footprint of the patient-heating element 102 (as suggested by the illustration). For many application settings, however, it will suffice if the electrosurgical return electrode 301 is considerably smaller than the patient-heating element 102.

When the electrosurgical return electrode 301 is disposed directly between the patient-heating element 102 and the patient-support substrate 101, the material and form of the electrosurgical return electrode 301 should be such that the electrosurgical return electrode 301 will not unduly interfere with the thermal path between the patient-heating element 102 and the patient.

FIG. 4 illustrates another example as regards the flexibility of these teachings. In this example the patient-heating element 102 is again disposed on the non-patient underside of the patient-support substrate 101. The electrosurgical return electrode 301, however, is disposed atop the patient-support substrate 101 (and in likely physical contact therewith).

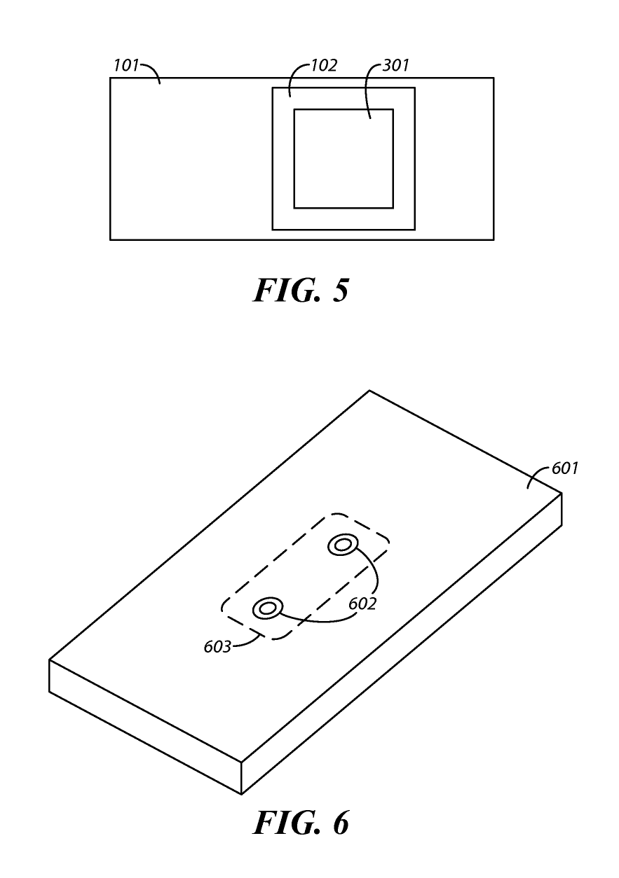

In all of these examples an integrated approach supports both active patient warming as well as electrosurgical support. As noted above the relative size of the various components can vary with the needs of a given application setting. FIG. 5 provides one illustrative example in these regards where the patient-heating element 102 is considerably smaller than the patient-support substrate 101 and where the electrosurgical return electrode 301 is, in turn, considerably smaller than the patient-heating element 102. Such a relative approach will likely be satisfactory for many purposes as the size of the return electrode contact area will usually not need to have as large a footprint as the heating area in order to provide satisfactory service.

Referring to FIG. 6, a cover 601 can serve to at least partially encompass the foregoing patient-support substrate 101, patient-heating element 102, and electrosurgical return electrode 301. This cover 601 can be permanently affixed or can be temporary and removable as desired. The cover can be comprised of a material that is impermeable to liquids and that is readily cleaned such as an appropriate supple plastic or an appropriately-treated fabric. This material can also be flame retardant if desired.

By one approach this cover 601 can comprise a multi-ply cover. In this case, for example, an outer ply can comprise a waterproof/flame-retardant material and an inner ply can comprise a stretch conductive fabric that is, for example, sewed to the outer ply. For example, the stretch conductive fabric offered by Less EMF, Inc. as part number 321 is a highly conductive elastic fiber fabric comprised seventy-six percent of medical grade plated silver plated and twenty-four percent Nylon elastic fiber fabric. Such a fabric comprises a knit conductive fabric having useful stretch properties. For example, using such a stretch fabric can serve to preserve the positive pressure reduction properties of a non-gel memory foam patient-support substrate 101 So configured, the inner ply can serve as the aforementioned electrosurgical return electrode.

By one approach the patient side of the cover 601 can include graphic elements 602 to indicate, for example, the location of the aforementioned temperature sensors 105. These graphic elements 602, in turn, can help a medical-services provider to properly locate and position a patient atop the patient-warming apparatus 100 to ensure proper warming of the patient. Similarly, one or more graphic elements 603 can serve to denote, for example, the boundaries of the electrosurgical return electrode 301 to again help facilitate properly placing the patient atop the patient-warming apparatus 100 to ensure a proper return path to the radio-frequency generator 107.

Those skilled in the art will recognize that a wide variety of modifications, alterations, and combinations can be made with respect to the above described embodiments without departing from the scope of the invention, and that such modifications, alterations, and combinations are to be viewed as being within the ambit of the inventive concept. As one illustrative example in these regards, a patient-support substrate 101 that comprises memory foam can be used in conjunction with an electrosurgical return path in the absence of a patient-heating element if desired. Such an approach can be useful in application settings that call for cauterization but no active patient warming. As another illustrative example in these regards, some or all of the patient-support substrate 101, patient-heating element 102, the electrosurgical return path, and/or the aforementioned sensors can be comprised, in whole or in part, of materials that are transparent or translucent to imaging energies (such as x-rays) to facilitate the use of high-energy imaging methodologies while also supporting patient warming and/or cauterization during a given treatment session.

We claim:

1. A patient-warming apparatus integrally comprising:
a patient-support substrate;
a patient-heating element disposed proximal to the patient-support substrate;
an electrosurgical return electrode disposed proximal to the patient-support substrate.

2. The patient-warming apparatus of claim 1 wherein the patient-support substrate comprises, at least in part, memory foam.

3. The patient-warming apparatus of claim 1 wherein the patient-heating element comprises a plurality of electrically-resistive traces disposed on a flexible substrate.

4. The patient-warming apparatus of claim 1 wherein the electrosurgical return electrode is integral to the patient-heating element.

5. The patient-warming apparatus of claim 1 wherein the electrosurgical return electrode is disposed between the patient-support substrate and the patient-heating element.

6. The patient-warming apparatus of claim 1 wherein the electrosurgical return electrode has a smaller footprint than the patient-heating element.

7. The patient-warming apparatus of claim 1 wherein the electrosurgical return electrode comprises a part of a capacitively-coupled electrosurgical return electrode.

8. The patient-warming apparatus of claim 1 further comprising:
at least one temperature sensor configured to sense a temperature of heat as delivered to a patient who is supported by the patient-support substrate.

9. The patient-warming apparatus of claim 8 further comprising:
a control circuit operably coupled to the at least one temperature sensor and configured to control the patient-heating element to compensate, at least in part, for heat exuded by the electrosurgical return electrode.

10. A method of forming a patient-warming apparatus comprising:
providing a patient-support substrate;
combining the patient-support substrate with both a patient-heating element and an electrosurgical return electrode.

11. The method of claim 10 wherein providing a patient-support substrate comprises providing a non-gel patient-support substrate.

12. The method of claim 11 wherein providing a non-gel patient-support substrate comprises providing a patient-support substrate comprised of memory foam.

13. The method of claim 10 wherein the patient-heating element and the electrosurgical return electrode share at least one common electricity-conveying component.

14. The method of claim 13 wherein the patient-heating element and the electrosurgical return electrode are formed integrally to one another.

15. The method of claim 10 wherein the patient-heating element and the electrosurgical return electrode are physically-separate discrete components.

16. The method of claim 10 further comprising:
providing at least one temperature sensor configured to sense a temperature that corresponds to heat being provided to a patient lying on the patient-support substrate.

17. The method of claim 16 further comprising:
providing a control circuit configured to operably couple to the at least one temperature sensor and to control the patient-heating element to compensate, at least in part, for heat exuded by the electrosurgical return electrode.
